(19) United States
(12) Patent Application Publication
(10) Pub. No.: US 2020/0368128 A1
(43) Pub. Date: Nov. 26, 2020

(54) CLEANING COMPOSITONS, KITS, AND METHODS INCLUDING GLUCAMINE DERIVATIVES

(71) Applicant: 3M INNOVATIVE PROPERTIES COMPANY, St. Paul, MN (US)

(72) Inventors: Richard P. Rusin, Woodbury, MN (US); Jie Yang, Woodbury, MN (US); Joel D. Oxman, Minneapolis, MN (US); Melinda B. Gustafson, Lake Elmo, MN (US)

(21) Appl. No.: 16/767,634

(22) PCT Filed: Nov. 26, 2018
(86) PCT No.: PCT/IB2018/059312

§ 371 Date: May 28, 2020

Related U.S. Application Data
(60) Provisional application No. 62/592,781, filed on Nov. 30, 2017.

(51) Int. Cl. A61K 8/41; C11D 1/29; C11D 3/10; C11D 3/30; C11D 17/00; C11D 17/06; A61K 8/19; A61Q 11/02; A61K 8/02

(52) U.S. Cl. CPC A61K 8/41; C11D 1/29; C11D 3/10; C11D 3/30; C11D 17/0073; C11D 17/06; A61K 2800/28; A61Q 11/02; A61K 8/022; A61K 2800/10; A61K 2800/88; A61K 2800/87; A61K 8/19

(57) ABSTRACT

Disclosed herein are cleaning compositions effective for cleaning an oral appliance or device. The cleaning composition can include: (i) a compound of Formula (I): wherein R¹ is a hydrogen atom or an alkyl group, R² is C(O)R³, R³ is a C₇-C₉ alkyl group, and n is an integer from 2 to 5; and (ii) a second component selected from the group consisting of persulfate (e.g., peroxymonosulfate and peroxydisulfate) salts, percarbonate salts, perborate salts, hypochlorite salts, dichloroisocyanurate salts, trichloroisocyanurate salts, tetraacetylethylenediamine, perphosphate salts, and combinations thereof. Optionally, the cleaning composition can further include water.

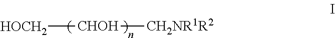

### BACKGROUND

The ability to remove biofilm from appliances and prevent biofilm formation on appliances have been longstanding and valued features in cleaning products, particularly those designed for removable oral appliances. Presently, cleaning has been attained by killing bacteria with antimicrobial agents. However, there is a growing recognition of the dangers of over-use of antimicrobials, due to the resulting evolution of resistant bacteria.

There is a continuing need for alternative approaches for cleaning appliances such as removable oral appliances.

### SUMMARY

In one aspect, the present disclosure provides cleaning compositions effective for cleaning an oral appliance or device.

In one embodiment, the cleaning composition includes: (i) a compound of Formula I:

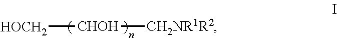

wherein R¹ is a hydrogen atom or an alkyl group, R² is C(O)R³, R³ is a C₇-C₉ alkyl group, and n is an integer from 2 to 5; and (ii) a second component selected from the group consisting of persulfate (e.g., peroxymonosulfate and peroxydisulfate) salts, percarbonate salts, perborate salts, hypochlorite salts, dichloroisocyanurate salts, trichloroisocyanurate salts, tetraacetylethylenediamine, perphosphate salts, and combinations thereof. Optionally, the cleaning composition further includes water.

In another embodiment, the composition includes active ingredients, the active ingredients including: (i) a compound of Formula I:

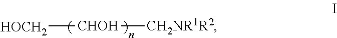

wherein R¹ is a hydrogen atom or an alkyl group, R² is C(O)R³, R³ is a C₇-C₉ alkyl group, and n is an integer from 2 to 5; and (ii) a second component selected from the group consisting of persulfate (e.g., peroxymonosulfate and peroxydisulfate) salts, percarbonate salts, perborate salts, hypochlorite salts, dichloroisocyanurate salts, trichloroisocyanurate salts, tetraacetylethylenediamine, perphosphate salts, and combinations thereof, wherein the active ingredients include 0.1 wt.-% to 99.9 wt.-% of the compound of Formula I, and 0.1 wt.-% to 99.9 wt.-% of the second component, based on the total weight of solids of the cleaning composition. It should be noted that a carrier or diluent, which can optionally be added to the cleaning composition, will not change the relative amounts based on solids of the compound of Formula I and the second component recited herein above.

In another aspect, the present disclosure provides methods for cleaning an oral appliance or device.

In one embodiment, the method includes: providing an oral appliance or device outside the oral cavity; and applying a cleaning composition as described herein to one or more surfaces of the oral appliance or device to clean the oral appliance or device.

In another aspect, the present disclosure provides kits that include a cleaning composition as described herein.

In one embodiment, the kit includes a cleaning composition as described herein; and instructions for using the kit. Optionally, the kit further includes an applicator.

In another embodiment, the kit includes a plurality of packages containing single use portions of a cleaning composition as described herein; and instructions for using the kit. In some embodiments, the plurality of packages includes 2 to 30 packages.

Moreover, at least in some embodiments, the compositions and methods described herein for cleaning appliances can advantageously inhibit or prevent biofilm formation on appliances as they reside in the oral environment, an advantage that current cleaning products do not provide.

As used herein, the term “appliance” is intended to be broadly interpreted to include removable oral appliances, intraoral dental devices, and invasive medical devices.

As used herein, removable oral appliances are functional treatment devices that a patient places and removes in their mouth periodically, Removable oral appliances include but are not limited to dentures (full and partial), orthodontic clear tray aligners, retainers, splints, mouthguards, night guards, sleep apnea devices, tongue thrust dental guard, NTI (Nociceptive Trigeminal Inhibition) appliances, TMJ devices, removable space maintainers, etc.

As used herein, intraoral dental devices are functional devices that are inserted into and removed from the oral cavity. Intraoral dental devices include intraoral cameras, intraoral 3D scanners, intraoral diagnostic devices, x-ray sensors, occlusal pressure sensors, dental curing lights, material delivery devices, etc. Of particular interest are devices that cannot undergo autoclave disinfection.

As used herein, invasive medical devices are functional devices that are inserted and removed from the body, either through a natural path such as mouth or nose, or through an incision. Invasive medical devices include catheters, laparoscopes, laparoscopic instruments, colonoscopes, stylets, ventilators, laryngoscope, endotracheal tubes, bronchoscope, and others.

As used herein, the term “compound” or “component” is a chemical substance which has a particular molecular identity or is made of a mixture of such substances, e.g., polymeric substances.

As used herein, the term “powder” refers to a dry, bulk solid composed of a large number of very fine particles that may flow freely when shaken or tilted.

As used herein, the term “particle” refers to a substance being a solid having a shape that can be geometrically determined. Particles can typically be analyzed with respect to, for example, grain size or diameter.

As used herein, the term “liquid” refers to any solvent or liquid that is able to at least partially disperse or dissolve a component at ambient conditions (e.g., 23° C.). A liquid typically has a viscosity below about 10 or below about 8 or below about 6 Pa*s.

As used herein, the phrases “room temperature” and “ambient temperature” are used interchangeably to mean a temperature in the range of 20° C. to 25° C.

As used herein, the term “biofilm” refers to a matrix including bacteria. Along with bacteria, a biofilm in the oral cavity of a subject can further include epithelial cells, leukocytes, macrophages, and other oral exudate.

As used herein, the phrase “biofilm inhibiting” refers to limiting the formation or growth of a biofilm.

As used herein, the term “alkyl” refers to a monovalent group that is a radical of an alkane, which is a saturated hydrocarbon. The alkyl group can be linear, branched, cyclic, or combinations thereof and can have 1 to 20 carbon atoms. In some embodiments, the alkyl group includes 1 to 18, 1 to 12, 1 to 10, 1 to 8, 1 to 6, or 1 to 4 carbon atoms. Examples of alkyl groups include, but are not limited to, methyl, ethyl, n-propyl, isopropyl, n-butyl, isobutyl, tert-butyl, n-pentyl, n-hexyl, cyclohexyl, n-heptyl, n-octyl, and ethylhexyl.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.”

The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain situations by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Also, as used herein in connection with a measured quantity, the term “approximately” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

The above brief description of various embodiments of the present disclosure is not intended to describe each embodiment or every implementation of the present disclosure. Rather, a more complete understanding of the disclosure will become apparent and appreciated by reference to the following description and claims in view of the accompanying drawings. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list. Further, it is to be understood that other embodiments may be utilized and structural changes may be made without departing from the scope of the present disclosure.

### DETAILED DESCRIPTION OF ILLUSTRATIVE EMBODIMENTS

In one aspect, the present disclosure provides cleaning compositions that can be effective for cleaning an oral appliance or device.

In one embodiment, the cleaning composition includes: (i) a compound of Formula I:

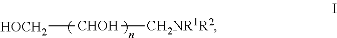

wherein R¹ is a hydrogen atom or an alkyl group, R² is C(O)R³, R³ is a C₇-C₉ alkyl group, and n is an integer from 2 to 5; and (ii) a second component selected from the group consisting of persulfate (e.g., peroxymonosulfate and peroxydisulfate) salts, percarbonate salts, perborate salts, hypochlorite salts (e.g., sodium hypochlorite, calcium hypochlorite), dichloroisocyanurate salts, trichloroisocyanurate salts, tetraacetylethylenediamine, perphosphate salts, and combinations thereof. It is understood that the second component of the cleaning composition is not typically present in compositions suitable for use (e.g., pharmaceutically acceptable and/or food grade) in the oral environment. Optionally, the cleaning composition can further include water. Optionally, the cleaning can further include one or more additional components including, but not limited to, hydrogen peroxide, peroxy acids, t-butyl hydroperoxide, peracetic acid, and benzoyl peroxide.

In another embodiment, the composition includes active ingredients, the active ingredients including: (i) a compound of Formula I:

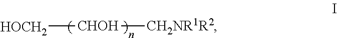

wherein R¹ is a hydrogen atom or an alkyl group, R² is C(O)R³, R³ is a C₇-C₉ alkyl group, and n is an integer from 2 to 5; and (ii) a second component selected from the group consisting of persulfate (e.g., peroxymonosulfate and peroxydisulfate) salts, percarbonate salts, perborate salts, hypochlorite salts, dichloroisocyanurate salts, trichloroisocyanurate salts, tetraacetylethylenediamine, perphosphate salts, and combinations thereof, wherein the active ingredients include 0.1 wt.-% to 99.9 wt.-% of the compound of Formula I, and 0.1 wt.-% to 99.9 wt.-% of the second component, based on the total weight of solids of the cleaning composition. It should be noted that a carrier or diluent, which can optionally be added to the cleaning composition, will not change the relative amounts based on solids of the compound of Formula I and the second component recited herein above.

Some embodiments of the composition can include at least 0.2, 0.5, 1, 2, 3, 4, 5, 10, or even 15 wt.-% of a compound of Formula I. Some embodiments of the composition can include up to 90, 85, 80, 70, 60, or even 50 wt.-% of a compound of Formula I. Some embodiments of the composition can include at least 0.2, 0.5, 1, 2, 3, 4, 5, 10, or even 15 wt.-% of the second component. Some embodiments of the composition can include up to 90, 85, 80, 70, 60, or even 50 wt.-% of the second component.

In the compound of Formula I, the group R¹ is a hydrogen atom or an alkyl group. When R¹ is an alkyl group, the alkyl group can include about one carbon atom (i.e., C₁), about two carbon atoms (i.e., C₂), about three carbon atoms (i.e., C₃), about four carbon atoms (i.e., C₄), about five carbon atoms (i.e., C₅), about six carbon atoms (i.e., C₆), about eight carbon atoms (i.e., C₈), about ten carbon atoms (i.e., C₁₀), about twelve carbon atoms (i.e., C₁₂), more than about twelve carbon atoms, or a range between and including any two of these values. For example, when R¹ is an alkyl group, R¹ can be a C₁-C₂ alkyl group, a C₁-C₁₀ alkyl group, C₁-C₆ alkyl group, or a C₂-C₇ alkyl group. In particular embodiments, R¹ is a hydrogen atom or a C₁-C₂ alkyl group. When R¹ is an alkyl group, the alkyl group can include a straight chain alkyl group, a branched alkyl group, or a cyclic alkyl group. Non-limiting examples of alkyl groups include methyl, ethyl, 1-propyl, isopropyl, butyl, isobutyl, sec-butyl, tert-butyl, pentyl, isopentyl, neopentyl, hexyl, heptyl, 2-ethylhexyl, octyl, isooctyl, nonyl, decyl, undecyl, dodecyl, tetradecyl, pentadecyl, octadecyl, cyclohexyl, 4-methylcyclohexyl, cyclohexylmethyl, cyclopentyl, and cyclooctyl.

In the compound of Formula I, the group R³ is a C₇-C₉ alkyl group, for example, an alkyl group including about seven carbon atoms (i.e., C₇), about eight carbon atoms (i.e., C₈), or about nine carbon atoms (i.e., C₉). Such alkyl groups can include a straight chain alkyl group, a branched alkyl group, or a cyclic alkyl group. Non-limiting examples of C₇-C₉ alkyl groups for R³ include those listed for R¹ (e.g., heptyl, 2-ethylhexyl, octyl, isooctyl, and nonyl). In particular embodiments, R³ is a C₈ alkyl group (e.g., octyl). It is understood that where R¹ is an alkyl group (rather than a hydrogen atom), such alkyl group can be the same or different from the C₇-C₉ alkyl group of R³.

In the compound of Formula I, n is an integer from about 2 to about 5. In some embodiments, n is an integer having a value of about 2, about 3, about 4, or about 5. In particular embodiments, n is an integer having a value of about 4. It is understood that the cleaning compositions of the present disclosure can include more than one compound of Formula I and that the compounds can be represented by Formula I with different integer values of n. In these embodiments, the average value of n of a compound can be a non-integer. In particular embodiments, a cleaning composition of the present disclosure can include only one compound of Formula I, and n is an integer having a value of 2, 3, 4, or 5.

Examples of persulfate (e.g., peroxymonosulfate and peroxydisulfate) salts include, but are not limited to, sodium persulfate, potassium persulfate, sodium peroxomonosulfate, ammonium persulfate, sodium peroxymonosulfate, potassium peroxymonosulfate, and combinations thereof.

Examples of percarbonate salts include, but are not limited to, sodium percarbonate, potassium percarbonate, and combinations thereof.

Examples of perborate salts include, but are not limited to, sodium perborate anhydrous, sodium perborate monohydrate, potassium perborate, magnesium perborate, and combinations thereof.

Examples of hypochlorite salts include, but are not limited to, sodium hypochlorite, calcium hypochlorite, potassium hypochlorite, and combinations thereof.

Examples of dichloroisocyanurate salts include, but are not limited to, sodium dichloroisocyanurate, potassium dichloroisocyanurate, magnesium di(dichloroisocyanurate), and combinations thereof.

Examples of trichloroisocyanurate salts include, but are not limited to, sodium trichloroisocyanurate, potassium trichloroisocyanurate, and combinations thereof. Examples of perphosphate salts include, but are not limited to, sodium perphosphate, barium perphosphate, calcium perphosphate, potassium perphosphate, ammonium perphosphate, and combinations thereof.

The cleaning composition may be provided in a variety of forms including, but not limited to, tablets, powder, granules, liquid or paste concentrates, ready-to-use liquids, and dispersions (aqueous or non-aqueous).

In some embodiments, the cleaning composition is a dry composition. In certain embodiments, the dry composition is a powder. In other certain embodiments, the dry composition is a tablet.

In some embodiments, the cleaning composition further includes 5 wt.-% to 99 wt.-% water, based on the total weight of the cleaning composition.

In some embodiments, the cleaning composition is a concentrate that can be diluted with water before use. In certain embodiments, the concentrate includes 0.1 wt.-% to 80 wt.-% water prior to dilution, based on the total weight of the concentrate.

In some embodiments, the cleaning composition is a ready for use composition. In certain embodiments, the ready for use composition includes 5 wt.-% to 99 wt.-% water, based on the total weight of the ready for use composition.

In some embodiments, the cleaning composition further includes a compound of Formula II or a pharmaceutically acceptable salt thereof:

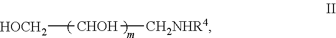

wherein R⁴ is a hydrogen atom or an alkyl group, m is an integer from 2 to 5.

In the compound of Formula II or a pharmaceutically acceptable salt thereof, R⁴ is a hydrogen atom or an alkyl group. When R⁴ is an alkyl group, the alkyl group can include about one carbon atom (i.e., C₁), about two carbon atoms (i.e., C₂), about three carbon atoms (i.e., C₃), about four carbon atoms (i.e., C₄), about five carbon atoms (i.e., C₅), about six carbon atoms (i.e., C₆), about eight carbon atoms (i.e., C₅), about ten carbon atoms (i.e., C₁₀), about twelve carbon atoms (i.e., C₁₂), more than twelve carbon atoms, or a range between and including any two of these values. For example, when R⁴ is an alkyl group, R⁴ can be a C₁-C₂ alkyl group, a C₁-C₁₀ alkyl group, a C₁-C₈ alkyl group, a C₁-C₆ alkyl group, or a C₂-C₇ alkyl group. In particular embodiments, R⁴ is a hydrogen atom, a C₁-C₁₂ alkyl group, a C₁-C₁₀ alkyl group, or a C₁-C₈ alkyl group. When R¹ is an alkyl group, the alkyl group can include a straight chain alkyl group, a branched alkyl group, or a cyclic alkyl group. Non-limiting examples of alkyl groups include methyl, ethyl, 1-propyl, isopropyl, butyl, isobutyl, sec-butyl, tert-butyl, pentyl, isopentyl, neopentyl, hexyl, heptyl, 2-ethylhexyl, octyl, isooctyl, nonyl, decyl, undecyl, dodecyl, tetradecyl, pentadecyl, octadecyl, cyclohexyl, 4-methylcyclohexyl, cyclohexylmethyl, cyclopentyl, and cyclooctyl.

In the compound of Formula II, or a pharmaceutically acceptable salt thereof, m is an integer from about 2 to about 5. In some embodiments, m is an integer having a value of about 2, about 3, about 4, or about 5. In particular embodiments, m is an integer having a value of about 4. It is understood that the cleaning compositions of the present disclosure can include more than one compound of Formula II or pharmaceutically acceptable salt thereof and that the compounds can be represented by Formula II (or a pharmaceutically acceptable salt thereof) with different integer values of m. In these embodiments, the average value of m can be a non-integer. In particular embodiments, the cleaning compositions of the present disclosure include only one compound of Formula II or a pharmaceutically acceptable salt thereof, and m is an integer having a value of 2, 3, 4, or 5.

The compound of Formula II can be in the form of a pharmaceutically acceptable salt. Pharmaceutically acceptable salts include ammonium salts. Thus, in some embodiments, a cleaning composition of the present disclosure can include an ammonium salt. An ammonium salt can be represented as the reaction product of an acid with an amine, or as the reaction product of an amine with an alkylating agent such as, for example, iodomethane, bromoethane, or benzyl bromide. An ammonium salt includes a protonated amine compound, for example a compound of Formula II in which the nitrogen atom of the group NHR⁴ has been protonated with an inorganic acid, an organic acid, or a combination of an inorganic acid and an organic acid. An ammonium salt also includes an alkylated amine compound, for example a compound of Formula II in which the nitrogen atom of the group NHR⁴ has been alkylated with an alkylating agent.

An ammonium salt includes at least one counter ion that can be an inorganic anion, an organic anion, or a combination of anions. A combination of anions includes a combination of more than one inorganic anion, a combination of more than one organic anion, or a combination of an inorganic ion and an organic anion. Inorganic ions include, for example, halide (fluoride, chloride, bromide, and iodide), nitrate, sulfate, phosphate (including one or more of phosphate, hydrogen phosphate, and dihydrogen phosphate inorganic ions), tetrafluoroborate, and tetra(aryl)borates. Tetra(aryl)borates include compounds having the formula Z₄B⁻, where Z is an aromatic group, for example a substituted or unsubstituted phenyl group. Examples of tetra(aryl)borates include, but are not limited to, tetraphenylborate, tetrakis(4-methylphenyl)borate, tetrakis(2-methylphenyl)borate, tetrakis(1,3,5-trimethylphenyl)borate, tetrakis(4-fluorophenyl)borate, tetrakis(pentafluorophenyl)borate, and tetrakis(4-trifluoromethylphenyl)borate. Organic anions include, for example, alkanoates (such as, for example, acetate, propionate, and butanoate), benzoate, fumarate, maleate, tartrate, ascorbate, benzenesulfonate, toluenesulfonate, and citrate.

In certain implementations, an ammonium salt can be formed by protonation of a compound of Formula II with an inorganic acid, an organic acid, or a combination of an inorganic acid and an organic acid. In another embodiment, an ammonium salt can be formed by alkylation of a compound of Formula II, with an alkylating agent. In yet another embodiment, an ammonium salt can be formed by an ion exchange or metathesis reaction with a previously formed ammonium salt.

In some embodiments, the compound of Formula II is in the form of a pharmaceutically acceptable salt, such as an ammonium halide salt, an ammonium phosphate salt (including one or more of an ammonium phosphate salt, an ammonium hydrogen phosphate salt, or an ammonium dihydrogen phosphate salt), or an ammonium citrate salt.

It is recognized that the compounds of Formulas I and II include chiral carbon atoms. For simplicity, in Formulas I and II, the stereochemical configuration about each of the chiral carbon atoms is not specified. It is intended that Formulas I and II, as used in this description and in the claims, represents each of the compounds having any of the possible stereochemical configurations. In some embodiments, the compounds of Formulas I and II are amino sugar alcohols and derivatives having the common names D-glucamine, N-methyl-D-glucamine, N-ethyl-D-glucamine, N-octyl-D-glucamine, and N-methyl-N-octanoyl-D-glucamine, N-methyl-N-nonanoyl-D-glucamine, N-methyl-N-decanoyl-D-glucamine.

In some embodiments, the combination of the compound of Formula I and the compound of Formula II or a pharmaceutically acceptable salt thereof may demonstrate improved efficacy in inhibiting biofilm formation on a surface, in comparison to the compound of Formula I alone. This synergistic effect is useful for the development of improved cleaning compositions as disclosed herein. In certain embodiments, the addition of the compound of Formula II or a pharmaceutically acceptable salt thereof to the cleaning care compositions described herein including the compound of Formula I, may provide for enhanced biofilm inhibition activity.

In some embodiments, the cleaning composition may include an abrasive. The abrasive can serve one or more functions including but not limited to abrading and/or otherwise cleaning debris and/or plaque from the appliance surface, removing stains from the appliance surface, polishing the appliance surface, and/or providing a whitening effect on the appliance surface.

Examples of suitable abrasives can include, but are not limited to, silica, silica gel, hydrated silica, precipitated silica, fused silica, alumina, calcined alumina, insoluble phosphates, calcium carbonate, ground glass, silicon carbide, ilmenite, sodium bicarbonate, bentonite, mica, zirconia, zirconia silicate, topaz, titanium dioxide, precipitated lime, chalk, pumice (e.g., flour of pumice), zeolites, talcum, kaolin, diatomaceous earth, silicates, glycine, resinous abrasives such as urea-formaldehyde condensation products, and the like. Among phosphates useful as abrasives are orthophosphates, polymetaphosphates and pyrophosphates, and salts thereof; illustrative examples are dicalcium orthophosphate dihydrate, dicalcium phosphate, calcium pyrophosphate, tricalcium phosphate, calcium polymetaphosphate, hydroxyapatite, magnesium orthophosphate, and insoluble sodium polymetaphosphate.

Also useful as an abrasive of the present disclosure is perlite, for example as described in U.S. Pat. No. 5,266,304 (Baffelli et al.) and U.S. Pat. No. 6,576,225 (Kilcher et al.).

In some embodiments, a water-soluble abrasive can be used (alone or in combination with other abrasives, each of which can be water soluble or water insoluble), such as described in U.S. Pat. No. 8,858,921 (Schmid et al.).

The average particle size of the abrasive is generally about 0.1 micrometers, about 1 micrometers, about 5 micrometers, about 10 micrometers, about 15 micrometers, about 20 micrometers, about 25 micrometers, about 30 micrometers, about 35 micrometers, about 40 micrometers, about 50 micrometers, about 75 micrometers, about 90 micrometers, about 100 micrometers, about 150 micrometers, about 200 micrometers, about 250 micrometers, about 300 micrometers, or a range between and including any two of these values. For example, in some embodiments, the average particle size of the abrasive is about 0.1 to about 300 micrometers, about 20 to about 300 micrometers, about 90 to about 300 micrometers, about 0.1 to about 50 micrometers, about 1 to about 40 micrometers, or about 5 to about 30 micrometers. The cleaning compositions of the present disclosure can include more than one abrasive. In such instances, the average particle size is intended to refer to the average particle size of each abrasive included in the cleaning composition. For example, a cleaning composition can include, as a first abrasive, silica with an average particle size of about 1 to about 40 micrometers and, as a second abrasive, calcium carbonate with an average particle size of about 30 to about 75 micrometers.

In certain implementations of the cleaning compositions of the present disclosure, silica-containing abrasives of various types are incorporated because they provide excellent cleaning and polishing performance, without unduly abrading the surface of the oral appliance. The silica-containing abrasive materials described herein, as well as other abrasives, generally have an average particle size ranging from about 0.1 to about 30 micrometers or from about 5 to about 15 micrometers. The silica-containing abrasive can be precipitated silica or silica gels such as the silica xerogels described in U.S. Pat. No. 3,538,230 (Pader et al.) and U.S. Pat. No. 3,862,307 (DiGuilio). Additional types of silica-containing abrasives useful in the cleaning compositions of the present disclosure are described in U.S. Pat. No. 4,340,583 (Wason), U.S. Pat. No. 5,589,160 (Rice), U.S. Pat. No. 5,603,920 (Rice), U.S. Pat. No. 5,651,958 (Rice), U.S. Pat. No. 5,658,553 (Rice), and U.S. Pat. No. 5,716,601 (Rice). Suitable silica-containing abrasives for inclusion in the cleaning compositions described herein are available from: Huber Engineered Materials under the tradename ZEODENT; Grace under the tradenames SYLOID and SYLODENT; and Evonik under the tradename SIDENT.

In certain embodiments, the cleaning composition includes sodium bicarbonate as an abrasive.

In some embodiments, the cleaning composition can optionally include one or more carriers, additives, adjuvants, agents, and/or modifiers. In some embodiments, the one or more carriers, additives, adjuvants, agents, and/or modifiers are selected from the group consisting of enzymes, peroxides, disinfectants, polymers, surfactants, solvents, anti-tartar agents, anti-calculus agents, preservatives, abrasives, rheology modifiers, buffering agents, diluents, solvents, fillers, emulsifiers, foaming agents, lubricants, pH modifying agents, humectants, antimicrobial agents, oils, flavors, fragrances, coloring agents, stabilizers, decorative agents, waxes, tetraacetylethylenediamine, and combinations thereof.

Any individual component of a cleaning composition of the present disclosure can be dissolved, dispersed, suspended, or emulsified in the cleaning composition. It is understood that while general attributes of each of the categories of components can differ; there can be some common attributes, and any given component can serve multiple purposes within two or more of such categories of components. For example, if sorbitol is included in the cleaning composition, sorbitol can act a sweetener and a humectant.

In various embodiments, cleaning compositions of the present disclosure can include a carrier. The carrier, if present, can include a liquid, a solid, or both. In some embodiments, the carrier can be a liquid at about room temperature. In other embodiments, the carrier can be a solid at about room temperature. It is understood that a plurality of carriers can be used. Examples of liquid carriers include, but are not limited to, water, glycerin, propylene glycol, polyalkylene glycols (e.g., polyethylene glycol, polypropylene glycol, etc.), and combinations thereof. Further examples of suitable carriers include those described in U.S. Pat. No. 6,669,929 (Boyd et al.), U.S. Pat. No. 6,379,654 (Gebreselassie et al.), and U.S. Pat. No. 4,894,220 (Nabi et al.). In some embodiments, the carrier (or combinations of carriers) is substantially free of water (e.g., less than about 5 wt.-%, or less than about 1 wt.-% water, based on the total weight of the carrier(s)). In some embodiments where the carrier or combinations of carriers are substantially free of water, such carrier or combination of carriers is water miscible (e.g., water soluble). In further embodiments including carriers (or combinations of carriers) which are substantially free of water, the cleaning composition is also substantially free of water (e.g., less than about 5 wt.-% water or less than about 1 wt.-% water, based on the total weight of the cleaning composition). In other embodiments, the cleaning composition includes water, and water is present in an amount of at least about 10 wt.-%, at least about 20 wt.-%, at least about 30 wt.-%, at least about 40 wt.-%, or at least about 50 wt.-%, based on the total weight of the cleaning composition.

Each non-carrier component of the cleaning composition, including but not limited to the compound of Formula I, the compound of Formula II or pharmaceutically acceptable salt thereof, and the abrasive, can independently be dissolved, dispersed, suspended, or emulsified in the carrier. In some embodiments, at least one component of the cleaning composition is dissolved in the carrier. In some embodiments, at least one component of the cleaning composition is dispersed in the carrier. In some embodiments, at least one component of the cleaning composition is suspended in the carrier. In some embodiments, at least one component of the cleaning composition is emulsified in the carrier. In some embodiments, the compound of Formula I and the compound of Formula II or a pharmaceutically acceptable salt thereof are each dissolved in the carrier.

In some embodiments, cleaning compositions of the present disclosure can include one or more antibacterial agents. Examples of suitable antibacterial agents can include, but are not limited to, aldehydes (glutaraldehyde, phthalaldehyde), salts of phenolics or acids, chlorhexidine or its derivatives (including acid adducts such as acetates, gluconates, chlorides, nitrates, sulfates or carbonates), and combinations thereof.

Non-limiting examples of antibacterial agents include: zinc salts, zinc oxide, tin salts, tin oxide, benzalkonium chloride, hexetidine, long chain alkyl ammonium or pyridinium salts (e.g., cetylpyridinium chloride, tetradecylpyridinium chloride), essential oils (e.g., thymol), furanones, chlorhexidine and salt forms thereof (e.g., chlorhexidine gluconate), sanguinarine, triclosan, stannous chloride, stannous fluoride, octenidine, nonionic or ionic surfactants (e.g., quaternary ammonium compounds), alcohols (monomeric, polymeric, mono-alcohols, poly-alcohols), aromatic alcohols (e.g., phenol)), antimicrobial peptides (e.g., histatins), bacteriocins (e.g., nisin), antibiotics (e.g., tetracycline), aldehydes (e.g., glutaraldehyde) inorganic and organic acids (e.g., benzoic acid, salicylic acid, fatty acids, etc.) or their salts, derivatives of such acids such as esters (e.g., p-hydroxybenzoates or other parabens, glycerol esters of fatty acids such as lauricidin), fluoride, EDTA, silver compounds, silver nanoparticles, peroxides (e.g., hydrogen peroxide), and combinations thereof. The antibacterial agent can be dissolved, dispersed, suspended, or emulsified in the cleaning composition. In some embodiments wherein the cleaning compositions include a carrier, the antibacterial agent can be dissolved, dispersed, suspended, or emulsified in the carrier. In other embodiments, the cleaning compositions are free of an antibacterial agent.

Some embodiments of cleaning compositions of the present disclosure can include one or more surfactants. The surfactant can be an anionic surfactant, a cationic surfactant, a zwitterionic surfactant, a nonionic surfactant, or a combination thereof. In some cases, the surfactant can also serve as a foaming agent. Examples of suitable surfactants and foaming agents can include, but are not limited to, sodium lauryl sulfate, as well as those described in U.S. Pat. No. 6,174,515 (Suhonen) and U.S. Pat. No. 4,301,141 (Stier et al.).

Cleaning compositions of the present disclosure can include one or more inorganic or a natural or synthetic thickeners or gelling agents. Optionally, one or more thickeners are present in a total amount of about 0.01 wt.-% to about 15 wt.-%, in some embodiments about 0.1 wt.-% to about 10 wt.-%, in some embodiments about 0.10 wt.-% to about 5 wt.-%, in some embodiments about 0.2 wt.-% to about 5 wt.-%, and in some embodiments about 0.2 wt.-% to about 1 wt.-%, based on the total weight of the cleaning compositions. In some embodiments, the proportions of thickeners in the cleaning compositions are sufficient to form an extrudable, shape-retaining product that can be squeezed from a tube. In some embodiments, the thickeners are sufficient to minimize splattering of the cleaning composition, if for example a rotating polishing device is used. In some embodiments, cleaning compositions of the present disclosure can include at least one thickener, useful for example to impart a desired consistency to the cleaning composition.

A wide variety of thickeners can be used. Suitable thickeners or gelling agents useful in the cleaning compositions of the present disclosure include amorphous silica (e.g., as available from Huber Corporation under the trade designation ZEODENT 165), fumed silica, precipitated silica, colloidal silica, natural and synthetic gums and colloids, poloxamers, carbomers, also known as carboxyvinyl polymers, carrageenan, Irish moss, iota-carrageenan, cellulosic polymers such as hydroxyethylcellulose, carboxymethylcellulose (carmellose, cellulose gum) and salts thereof, e.g., carmellose sodium, natural gums such as karaya, xanthan, gum Arabic, gum tragacanth, polyvinylpyrrolidone, agar, colloidal magnesium aluminum silicate, and combinations thereof. One or more thickeners are optionally present in a total amount of about 0.01 wt.-% to about 15 wt.-%, for example about 0.1 wt.-% to about 10 wt.-% or about 0.2 wt.-% to about 5 wt.-%, based on the total weight of the cleaning composition. The thickener or gelling agent can be dissolved, dispersed, suspended, or emulsified in the cleaning composition. In some embodiments wherein the cleaning compositions include a carrier, the thickener or gelling agent can be dissolved, dispersed, suspended, or emulsified in the carrier.

In some embodiments, cleaning compositions of the present disclosure can include a humectant or a plurality of humectants. Examples of humectants include, but are not limited to, polyhydric alcohols such as glycerin, sorbitol, xylitol, alkylene glycols (e.g., polyethylene glycol, propylene glycol), and combinations thereof. In various embodiments, humectants are operable to prevent hardening of the cleaning compositions upon exposure to air. In various embodiments humectants can also function as sweeteners. One or more humectants are optionally present, in a total amount, of about 1 wt.-%, about 2 wt.-%, about 5 wt.-%, about 10 wt.-%, about 15 wt.-%, about 20 wt.-%, about 25 wt.-%, about 30 wt.-%, about 40 wt.-%, about 50 wt.-%, about 60 wt.-%, about 70 wt.-%, about 80 wt.-%, greater than about 80 wt.-%, or at a range between and including any two of these values. For example, the cleaning compositions can include about 1 wt.-% to about 80 wt.-%, about 2 wt.-% to about 25 wt.-%, or about 5 wt.-% to about 15 wt.-%, based on the total weight of the cleaning composition. In some embodiments, the cleaning compositions include a humectant in an amount of at least about 10 wt.-%, at least about 15 wt.-%, or at least about 20 wt.-%, based on the total weight of the cleaning composition. In some such embodiments, the humectant includes sorbitol. The humectant can be dissolved, dispersed, suspended, or emulsified in the cleaning composition. In some embodiments wherein the cleaning compositions include a carrier, the humectant can be dissolved, dispersed, suspended, or emulsified in the carrier.

In some embodiments, cleaning compositions of the present disclosure can include a buffering agent or a buffering system, or a plurality of buffering agents or systems. Examples of suitable buffering agents include phosphate buffers as further described in U.S. Pat. No. 9,149,661 (Pilch et al.). The buffering agent or buffering system can be dissolved, dispersed, suspended, or emulsified in the cleaning composition. In some embodiments wherein the cleaning compositions include a carrier, the buffering agent or buffering system can be dissolved, dispersed, suspended, or emulsified in the carrier.

In some embodiments, cleaning compositions of the present disclosure can include a coloring agent or a plurality of coloring agents. The coloring agent can be any dye or pigment. The coloring agent can be dissolved, dispersed, suspended, or emulsified in the cleaning composition. In some embodiments wherein the cleaning compositions include a carrier, the coloring agent can be dissolved, dispersed, suspended, or emulsified in the carrier.

In some embodiments, cleaning compositions of the present disclosure can include a flavoring agent or a plurality of flavoring agents. In some embodiments, the flavoring agent includes an agent that imparts a flavor, e.g., a mint flavor, to the cleaning composition. Examples of suitable flavoring agents include, but are not limited to, oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl para-tert-butylphenylacetate, and mixtures thereof. The flavoring agent can be dissolved, dispersed, suspended, or emulsified in the cleaning composition. In some embodiments wherein the cleaning compositions include a carrier, the flavoring agent can be dissolved, dispersed, suspended, or emulsified in the carrier. The cleaning composition can include from about 0.001 wt.-% to about 5 wt.-% of the flavoring agent, based on the total weight of the cleaning composition.

Some embodiments may include one or more of the following additives: bicarbonate salts (e.g., sodium, potassium, and/or magnesium salts), carbonate salts (e.g., sodium and/or potassium salts), sulfate salts (e.g., sodium, potassium, and/or magnesium salts), salts of polyphosphates (e.g., sodium hexametaphosphate and/or sodium tripolyphosphate), hydroxides (e.g., sodium, potassium, and/or magnesium hydroxides), carboxylic acids (e.g., oxalic acid, malonic acid, malic acid, maleic acid, citric acid, and/or ethylenediaminetetraacetic acid), phosphate salts (e.g., sodium, potassium, and/or magnesium salts), enzymes, peroxides (e.g., hydrogen peroxide, urea peroxide, and/or sodium peroxide).

Some embodiments may include polymers or copolymers (e.g., polyvinylpyrrolidone/vinyl acetate (VP/VA) copolymer, itaconic/acrylic acid copolymer, and/or polyvinyl methyl ether/maleic acid (PVM/MA) copolymer).

Some embodiments may include a tablet binder, which is any substance that can be mixed with the other components of the dental appliance cleaning composition and pressed into a tablet form. Suitable binding substances include, but are not limited to, citric acid, polyvinylpyrrolidone, polyethylene glycol, lactose powder, sucrose powder, tapioca starch (cassava flour), and/or cellulose. When used, in some embodiments the tablet binder is present at about 0.1% to about 10% by weight, and in certain embodiments at about 0.2% to about 3% by weight, based on the total weight of the cleaning composition.

Some embodiments may include a lubricant, which is any substance that can be mixed with the other components of the dental appliance cleaning composition prior to tablet pressing in order to facilitate ejection of the tablet from the die. Suitable lubricants include, but are not limited to, magnesium stearate, stearic acid, magnesium lauryl sulfate, and/or sodium lauryl sulfate. When used, in some embodiments the lubricant is present at about 0.1% to about 5% by weight, and in certain embodiments at about 1% to about 3% by weight, based on the total weight of the cleaning composition.

In another aspect, the present disclosure provides methods for cleaning an oral appliance or device.

In one embodiment, the method includes: providing an oral appliance or device outside the oral cavity; and applying a cleaning composition as described herein to one or more surfaces of the oral appliance or device to clean the oral appliance or device.

In certain embodiments, the cleaning composition is a dry composition or concentrate, and applying the cleaning composition to the one or more surfaces of the oral appliance or device further includes adding water to the cleaning composition prior to and/or during cleaning of the oral appliance or device.

In certain embodiments, the cleaning composition is preloaded in an application device. In some certain embodiments, the application device is selected from the group consisting of a sprayer, a brush, a sponge, and combinations thereof.

In some embodiments, applying can include a method selected from the group consisting of spraying, brushing, sponging, wiping, swabbing, partially or totally immersing, rinsing, and combinations thereof.

In some embodiments, the method for cleaning the oral appliance or device can include partially or totally immersing the oral appliance or device in the cleaning composition for a time effective to adequately clean the oral appliance or device. The time can be up to about one second, up to about five seconds, up to about ten seconds, up to about thirty seconds, up to about one minute, up to about two minutes, up to about five minutes, up to about ten minutes, up to about fifteen minutes, up to about thirty minutes, or up to about sixty minutes. The time can be less than about one month, less than about two weeks, less than about one week, less than about twenty-four hours, less than about twenty hours, less than about sixteen hours, less than about twelve hours, less than about ten hours, less than about eight hours, less than about six hours, less than about four hours, or less than about two hours. Certain exemplary times include 30 seconds to 24 hours.

In some embodiments, the method for cleaning the oral appliance or device can include brushing, sponging, or wiping the oral appliance or device during and/or after application of the cleaning solution.

In some embodiments, the method for cleaning the oral appliance or device can further include rinsing the oral device or appliance after cleaning.

In another aspect, the present disclosure provides kits that include a cleaning composition as described herein.

In one embodiment, the kit includes a cleaning composition as described herein; and instructions for using the kit. Optionally, the kit further includes an applicator.

In another embodiment, the kit includes a plurality of packages containing single use portions of a cleaning composition as described herein; and instructions for using the kit. In some embodiments, the plurality of packages includes 2 to 30 packages.

### ILLUSTRATIVE EMBODIMENTS OF THE PRESENT DISCLOSURE

Various embodiments are disclosed that can provide cleaning compositions and kits, and methods of using same.

Embodiment 1A is a cleaning composition for cleaning an oral appliance or device, the cleaning composition comprising: (i) a compound of Formula I:

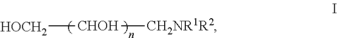

wherein R¹ is a hydrogen atom or an alkyl group, R² is C(O)R³, R³ is a C₇-C₉ alkyl group, and n is an integer from 2 to 5; and (ii) a second component selected from the group consisting of persulfate (e.g., peroxymonosulfate and peroxydisulfate) salts, percarbonate salts, perborate salts, hypochlorite salts, dichloroisocyanurate salts, trichloroisocyanurate salts, tetraacetylethylenediamine, perphosphate salts, and combinations thereof.

Embodiment 2A is the cleaning composition of embodiment 1A, wherein the cleaning composition further comprises water.

Embodiment 3A is the cleaning composition of embodiment 1A or 2A, wherein the cleaning composition further comprises a compound of Formula II or a pharmaceutically acceptable salt thereof:

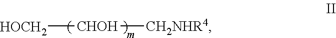

wherein R⁴ is a hydrogen atom or an alkyl group, m is an integer from 2 to 5.

Embodiment 4A is the cleaning composition of any one of embodiments 1A to 3A, wherein the cleaning composition further comprises an abrasive.

Embodiment 5A is the cleaning composition of embodiment 4A, wherein the abrasive is sodium bicarbonate.

Embodiment 6A is the cleaning composition of any one of embodiments 1A to 5A, wherein the cleaning composition further comprises one or more carriers, additives, adjuvants, agents, and/or modifiers.

Embodiment 7A is the cleaning composition of embodiment 6A, wherein the one or more carriers, additives, adjuvants, agents, and/or modifiers are selected from the group consisting of enzymes, peroxides, disinfectants, polymers, surfactants, solvents, anti-tartar agents, anti-calculus agents, preservatives, abrasives, rheology modifiers, buffering agents, diluents, solvents, fillers, emulsifiers, foaming agents, lubricants, pH modifying agents, humectants, antimicrobial agents, oils, flavors, fragrances, coloring agents, stabilizers, decorative agents, waxes, tetraacetylethylenediamine, and combinations thereof.

Embodiment 1B is a cleaning composition for cleaning an oral appliance or device, the cleaning compositon comprising active ingredients, the active ingredients comprising:

(i) a compound of Formula I:

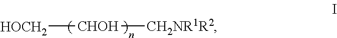

wherein R¹ is a hydrogen atom or an alkyl group, R² is C(O)R³, R³ is a C₇-C₉ alkyl group, and n is an integer from 2 to 5; and (ii) a second component selected from the group consisting of persulfate (e.g., peroxymonosulfate and peroxydisulfate) salts, percarbonate salts, perborate salts, hypochlorite salts, dichloroisocyanurate salts, trichloroisocyanurate salts, tetraacetylethylenediamine, perphosphate salts, and combinations thereof, wherein the active ingredients comprise 0.1 wt.-% to 99.9 wt.-% of the compound of Formula I, and 0.1 wt.-% to 99.9 wt.-% of the second component, based on the total weight of solids of the cleaning composition.

Embodiment 2B is the cleaning composition of embodiment 1B, wherein the cleaning composition is a dry composition.

Embodiment 3B is the cleaning composition of embodiment 2B, wherein the dry composition is a powder.

Embodiment 4B is the cleaning composition of embodiment 2B, wherein the dry composition is a tablet.

Embodiment 5B is the cleaning composition of embodiment 1B, wherein the cleaning composition further comprises 5 wt.-% to 99 wt.-% water, based on the total weight of the cleaning composition.

Embodiment 6B is the cleaning composition of any one of embodiments 1B to 5B, wherein the cleaning composition is a concentrate that can be diluted with water before use.

Embodiment 7B is the cleaning composition of embodiment 6B, wherein the concentrate comprises 0.1 wt.-% to 80 wt.-% water prior to dilution, based on the total weight of the concentrate.

Embodiment 8B is the cleaning composition of embodiment 5B wherein the cleaning composition is a ready for use composition.

Embodiment 9B is the cleaning composition of embodiment 8B, wherein the ready for use composition comprises 5 wt.-% to 99 wt.-% water, based on the total weight of the ready for use composition.

Embodiment 10B is the cleaning composition of any one of embodiments 1B to 9B, wherein the cleaning composition further comprises a compound of Formula II or a pharmaceutically acceptable salt thereof:

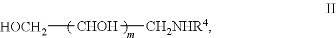

wherein R⁴ is a hydrogen atom or an alkyl group, m is an integer from 2 to 5.

Embodiment 11B is the cleaning composition of any one of embodiments 1B to 10B, wherein the cleaning composition further comprises an abrasive.

Embodiment 12B is the cleaning composition of embodiment 11B, wherein the abrasive is sodium bicarbonate.

Embodiment 13B is the cleaning composition of any one of embodiments 1B to 12B, wherein the cleaning composition further comprises one or more carriers, additives, adjuvants, agents, and/or modifiers.

Embodiment 14B is the cleaning composition of embodiment 13B, wherein the one or more carriers, additives, adjuvants, agents, and/or modifiers are selected from the group consisting of enzymes, peroxides, disinfectants, polymers, surfactants, solvents, anti-tartar agents, anti-calculus agents, preservatives, abrasives, rheology modifiers, buffering agents, diluents, solvents, fillers, emulsifiers, foaming agents, lubricants, pH modifying agents, humectants, antimicrobial agents, oils, flavors, fragrances, coloring agents, stabilizers, decorative agents, waxes, tetraacetylethylenediamine, and combinations thereof.

Embodiment 1C is a method for cleaning an oral appliance or device, the method comprising: providing an oral appliance or device outside the oral cavity; and applying a cleaning composition according to any one of embodiments 1A to 7A or 1B to 14B to one or more surfaces of the oral appliance or device to clean the oral appliance or device.

Embodiment 2C is the method of embodiment 1C, wherein the cleaning composition is a dry composition or concentrate, and applying the cleaning composition to the one or more surfaces of the oral appliance or device further comprises adding water to the cleaning composition prior to and/or during cleaning of the oral appliance or device.

Embodiment 3C is the method of embodiment 1C or 2C, wherein the cleaning composition is preloaded in an application device.

Embodiment 4C is the method of embodiment 3C, wherein the application device is selected from the group consisting of a sprayer, a brush, a sponge, and combinations thereof.

Embodiment 5C is the method of embodiment 4C, wherein applying comprises a method selected from the group consisting of spraying, brushing, sponging, and combinations thereof.

Embodiment 6C is the method of any one of embodiments 1C to 5C, wherein applying the cleaning composition comprises immersing the oral appliance or device in the cleaning composition for a time effective to adequately clean the oral appliance or device.

Embodiment 7C is the method of embodiment 6C, wherein the time is 30 seconds to 24 hours.

Embodiment 8C is the method of any one of embodiments 1C to 7C, wherein the method further comprises brushing, sponging, or wiping the oral appliance or device during and/or after application of the cleaning solution.

Embodiment 9C is the method of any one of embodiments 1C to 8C, wherein the method further comprises rinsing the oral device or appliance after cleaning.

Embodiment 1D is a kit comprising: a cleaning composition according to any one of embodiments 1A to 7A or 1B to 14B; and instructions for using the kit.

Embodiment 2D is the kit of embodiment 1D, wherein the kit further comprises an applicator.

Embodiment 1E is a kit comprising: a plurality of packages containing single use portions of a cleaning composition according to any one of embodiments 1A to 7A or 1B to 14B; and instructions for using the kit.

Embodiment 2E is the kit of embodiment 1E, wherein the plurality of packages comprises 2 to 30 packages.

Objects and advantages of this disclosure are further illustrated by the following non-limiting examples, but the particular materials and amounts thereof recited in these examples, as well as other conditions and details, should not be construed to unduly limit this disclosure.

### EXAMPLES

As used herein, all parts and percentages are by weight, all water is deionized water (“DI water”), and all molecular weights are average molecular weights, unless otherwise specified. Unless otherwise specified, materials can be obtained from Sigma-Aldrich, Milwaukee, Wis.

D-Glucamine is commercially available from TCI America (Portland, Oreg.). N-Methyl-D-glucamine and N-ethyl-D-glucamine are commercially available from MP Biomedicals (Solon, Ohio). N-methyl-N-nonanoyl-D-glucamine (“MEGA-9”) is commercially available from EMD Chemicals, Inc. (San Diego, Calif.).

### Examples Ex. 1 Ex. 6

The following examples of cleaning compositions according to the present disclosure were mixed together in NALGENE bottles in weight/weight percentages (wt./wt/%) of the components listed in Table 1.

TABLE 1

| Material name | Ex 1 | Ex 2 | Ex 3 | Ex 4 | Ex 5 | Ex 6 |
|---|---|---|---|---|---|---|
| Water | 93.5% | 94.0% | 93.5% | 93.5% | 94.0% | 94.0% |
| MEGA-9 | 2.0% | 2.0% | 2.0% | 2.0% | 2.0% | 2.0% |
| NaHCO₃ | 2.0% | 2.0% | 2.0% | 2.0% | — | — |
| (Sodium Bicarbonate) | | | | | | |
| KHSO₅ | 1.0% | 0.5% | 1.0% | 1.0% | — | — |
| (Potassium | | | | | | |
| monopersulfate) | | | | | | |
| Na₂CO₃ | 0.5% | 0.5% | 0.5% | — | 1.0% | 1.0% |
| (Sodium Carbonate) | | | | | | |
| sodium percarbonate | 0.5% | 0.5% | — | — | — | — |
| tetraacetylethyl- | 0.5% | — | 0.5% | — | — | — |
| enediamine | | | | | | |
| sodium | — | 0.5% | 0.5% | — | — | 1.0% |
| hexametaphosphate | | | | | | |
| sodium perborate | — | — | — | 0.5% | — | — |
| sodium sulfate | — | — | — | 0.5% | 1.0% | 1.0% |
| disodium EDTA | — | — | — | 0.5% | — | — |
| sodium | — | — | — | — | 1.0% | — |
| tripolyphosphate | | | | | | |
| sodium | — | — | — | — | 1.0% | 1.0% |
| dichloroisocyanurate | | | | | | |
| total solids | 6.5% | 6.0% | 6.5% | 6.5% | 6.0% | 4.0% |
| total | 100.0% | 100.0% | 100.0% | 100.0% | 100.0% | 100.0% |

All cited references, patents, or patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. A cleaning composition for cleaning an oral appliance or device, the cleaning composition comprising:
(i) a compound of Formula I: 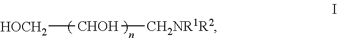
wherein R¹ is a hydrogen atom or an alkyl group, R² is C(O)R³, R³ is a C₇-C₉ alkyl group, and n is an integer from 2 to 5; and
(ii) a second component selected from the group consisting of persulfate salts, percarbonate salts, perborate salts, hypochlorite salts, dichloroisocyanurate salts, trichloroisocyanurate salts, tetraacetylethylenediamine, perphosphate salts, and combinations thereof.

2. The cleaning composition of claim 1, wherein the cleaning composition further comprises water.

3. A cleaning composition for cleaning an oral appliance or device, the cleaning composition comprising active ingredients, the active ingredients comprising:
(i) a compound of Formula I: 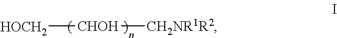
wherein R¹ is a hydrogen atom or an alkyl group, R² is C(O)R³, R³ is a C₇-C₉ alkyl group, and n is an integer from 2 to 5; and
(ii) a second component selected from the group consisting of persulfate salts, percarbonate salts, perborate salts, hypochlorite salts, dichloroisocyanurate salts, trichloroisocyanurate salts, tetraacetylethylenediamine, perphosphate salts, and combinations thereof,
wherein the active ingredients comprise 0.1 wt.-% to 99.9 wt.-% of the compound of Formula I, and 0.1 wt.-% to 99.9 wt.-% of the second component, based on the total weight of solids of the cleaning composition.

4. (canceled)

5. The cleaning composition of claim 3, wherein the composition is a powder or a tablet.

6. (canceled)

7. The cleaning composition of claim 3, wherein the cleaning composition further comprises 0.1 wt.-% to 99 wt.-% water, based on the total weight of the cleaning composition.

8.-11. (canceled)

12. The cleaning composition of claim 1, wherein the cleaning composition further comprises a compound of Formula II or a pharmaceutically acceptable salt thereof: 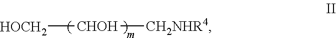
wherein R⁴ is a hydrogen atom or an alkyl group, m is an integer from 2 to 5.

13. The cleaning composition of claim 1, wherein the cleaning composition further comprises an abrasive.

14. The cleaning composition of claim 13, wherein the abrasive is sodium bicarbonate.

15. The cleaning composition of claim 1, wherein the cleaning composition further comprises one or more carriers, additives, adjuvants, agents, and/or modifiers.

16. (canceled)

17. A method for cleaning an oral appliance or device, the method comprising:
providing an oral appliance or device outside the oral cavity;
contacting a cleaning composition of claim 1 to one or more surfaces of the oral appliance or device; and
allowing the cleaning composition to contact the one or more surfaces for a period,
wherein the cleaning composition is effective to clean the oral appliance or device.

18. The method of claim 17, further comprising diluting the cleaning composition with water prior to and/or during the contacting of the cleaning composition to the one or more surfaces of the oral appliance or device.

19. The method of claim 17, wherein the cleaning composition is preloaded in an application device.

20. The method of claim 19, wherein the application device is selected from a sprayer, a brush, a sponge, and a combination thereof.

21. The method of claim 20, wherein contacting comprises spraying, brushing, sponging, and
a combination thereof, or
the contacting comprises at least partially immersing the oral applicant or device in the cleaning composition.

22. (canceled)

23. The method of claim 22, wherein the period is 30 seconds to 24 hours.

24. The method of claim 17, wherein the method further comprises brushing, sponging, or wiping the oral appliance or device during and/or after application of the cleaning solution.

25. The method of claim 17, wherein the method further comprises rinsing the oral device or appliance after cleaning.

26. A kit comprising:
a cleaning composition of claim 1; and
a set of instructions directing a user to:
provide an oral application or device outside the oral cavity;
contact the cleaning composition to one or more surfaces of the oral appliance or device; and
allow the cleaning composition to contact the one or more surfaces for a period.

27. The kit of claim 26, further comprising an applicator.

28. (canceled)

29. The kit of claim 26, wherein the cleaning composition is present in a plurality of packages.
